(19) United States
(12) Patent Application Publication
(10) Pub. No.: US 2024/0325316 A1
(43) Pub. Date: Oct. 3, 2024

(54) Encapsulation of Non-Steroidal Anti-Inflammatory Drugs (NSAIDs)

(71) Applicant: PRECISIO BIOTIX THERAPEUTICS, INC., Dover, DE (US)

(72) Inventors: Nancy Tawil, Cantley (CA); Simon De Grandpré, Montreal (CA)

(21) Appl. No.: 18/600,476

(22) Filed: Mar. 8, 2024

Related U.S. Application Data
(60) Provisional application No. 63/489,345, filed on Mar. 9, 2023.

(51) Int. Cl. A61K 9/51; A61K 31/192

(52) U.S. Cl. CPC A61K 9/5153; A61K 9/5138; A61K 9/5192; A61K 31/192

(57) ABSTRACT

A composition for delivering at least one Non-Steroidal Anti-Inflammatory Drug (NSAID) to a subject, the composition including biodegradable nanospheres, wherein the biodegradable nanospheres include the NSAID dispersed in a biodegradable polymer.

### FIELD OF THE INVENTION

The present invention relates to the general field of conditions involving inflammation, such as arthritis, among others, and pain treatment and is more particularly concerned with encapsulation of Non-Steroidal Anti-Inflammatory Drugs (NSAIDs).

### BACKGROUND

Rheumatoid diseases are a regrouping of illnesses that vary in their causes but result ultimately in cartilage damage and loss, often associated with pain, inflammation, and reduced motion of the affected joints. Physiologically, there is an alteration in the joint environment, compromising the homeostasis between cartilage wear and cartilage neosynthesis that is normally achieved in a healthy individual. The CDC estimates that arthritis and related conditions currently affect approximately 25% of the population of the United States of America.

There is no current pharmaceutical cure for arthritis and related conditions. For this reason, efforts are concentrated on treatment of the symptoms, which are mostly inflammation and pain. The most common pharmaceutical treatment is the prescription of Non-Steroidal Anti-Inflammatory Drugs (NSAIDs). Ibuprofen (IBU) is an NSAID routinely used as a pain killer, possessing anti-inflammatory properties when taken in sufficient amount. Oral administration of IBU suffers from systemic dispersion, where the dose required to achieve efficient concentration at the site of injury is relatively high. This systemic exposure correlates to some notable health issues such as peptic ulcers and associated bleeding, increased risk of cardiac incidents and nephrotoxicity. These side effects may be exacerbated when treatment is carried over prolonged periods of time. Therefore, there needs to be a way to administer the efficient dose locally. Intravenous or intramuscular delivery allows for local administration but is an intrusive and complicated procedure for a treatment that may require multiple daily injections due to the molecule's short half-life. Topical delivery remains as the simplest way to administer local efficient dosage.

Accordingly, there exists also a need for new methods and media for delivering NSAIDs. An object of the present invention is to provide such methods and media.

### SUMMARY OF THE INVENTION

In a broad aspect, there is provided a method to generate nanoparticles containing a relatively high amount of NSAIDs with appropriate morphology, size, encapsulation rate, stability, and release profile for the delivery of NSAIDs to a subject. There is also provided a method comprising applying to the skin of the subject a composition including the NSAIDs encapsulated in biodegradable nanoparticles.

In a broad aspect, there is provided a method of delivering at least one NSAID to a subject, the method comprising applying on a skin of the subject a composition including biodegradable nanospheres, wherein the biodegradable nanospheres include the at least one NSAID dispersed in a polymer.

Polymeric nanoparticles may allow for the diffusion of the NSAIDs at the site of action and controlled release of effective clinical dose for a prolonged period of time. Such nanoparticles may be used for the delivery of ibuprofen and NSAIDs in amino polymeric nanoparticles for localized topical treatment of pain and in inflammatory articular diseases, among others.

There may also be provided a method wherein the composition is provided to treat a condition selected from the group consisting of ankylosing spondylitis, antiphospholipid antibody syndrome, autoimmune encephalitis, chronic recurrent multifocal osteomyelitis, gout, Henoch-Schonlein purpura, juvenile dermatomyositis, juvenile idiopathic arthritis, juvenile lupus (SLE), juvenile scleroderma, juvenile vasculitis, Kawasaki disease, lupus (Systemic Lupus Erythematosus), mixed connective tissue disease, myositis, poststreptococcal inflammatory syndromes, psoriatic arthritis, reactive arthritis, rheumatoid arthritis, scleroderma, Sjogren's syndrome, spondyloarthritis/spondyloarthropathy, systemic juvenile idiopathic Arthritis, undifferentiated connective tissue disease, uveitis and vasculitis. among others.

There may also be provided a method wherein the NSAID is selected from the group consisting of diclofenac, diflunisal, etodolac, fenoprofen, flurbiprofen, ibuprofen, indomethacin, ketoprofen, ketorolac, mefenamic acid, meloxicam, nabumetone, naproxen, oxaprozin, piroxicam, sulindac, tolmetin and coxibs. These NSAIDs can be used in other embodiments of the present invention mentioned in this patent application.

There may also be provided a method wherein the composition is in the form of a cream or a formulation applicable using a roll-on applicator.

In another broad aspect, there is provided a composition comprising at least one NSAID encapsulated in biodegradable polymeric nanospheres. The NSAID is at least partially dispersed in the polymer itself. In these latter cases, the nanospheres may be hollow or full. Typically, the nanospheres are full, that is no central cavity is formed in them.

There may also be provided a composition wherein the biodegradable polymeric nanospheres include a polymer selected from
(1) a poly (ester amide urea) wherein at least one diol, at least one diacid, and at least one amino acid are linked together through an ester bond, an amide bond, and a urea bond,
(2) a poly (ester urethane urea) wherein at least one diol and at least one amino acid are linked together through an ester bond, a urethane bond, and a urea bond,
(3) a poly (ester amide urethane urea) wherein at least one diol, at least one diacid, and at least one amino acid are linked together through an ester bond, an amide bond, a urethane bond, and a urea bond,
(4) a poly (ester amide urethane) wherein at least one diol, at least one diacid, and at least one amino acid are linked together through an ester bond, an amide bond, and a urethane bond,
(5) a poly (ester urea) wherein at least one diol and at least one amino acid are linked together through an ester bond and a urea bond, and
(6) a poly (ester urethane) wherein at least one diol and at least one amino acid are linked together through an ester bond and a urethane bond,
further wherein
the at least one diol is a compound of formula:
HO—R₁—OH, R₁ is chosen from C₂-C₁₂ alkylene optionally interrupted by at least one oxygen, C₃-C₈ cycloalkylene, C₃-C₁₀ cycloalkylalkylene,

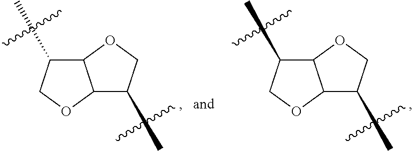

the at least one diacid is a compound of formula:

HO—(CO)—R₃—(CO)—OH,R₃ is C₂-C₁₂alkylene,

the at least one amino acid is chosen from naturally occurring amino acids and non-naturally occurring amino acid.

There may also be provided a composition wherein the polymer is a poly (ester amide urea) (PEAU) comprising the following two blocks with random distribution thereof:

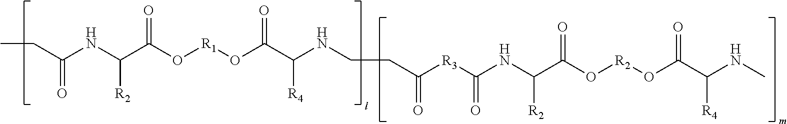

wherein
the ratio of l:m ranges from 0.05:0.95 to 0.95:0.05, l+m=1,
   R₁ is chosen from C₂-C₁₂ alkylenes optionally interrupted by at least one oxygen, C₃-C₈ cycloalkylenes, C₃-C₁₀ cycloalkylalkylenes,

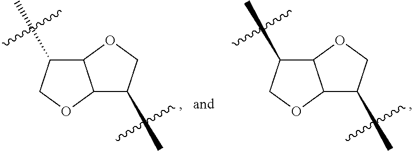

R₃ is C₂-C₁₂ alkylene,
R₂ and R₄ are independently chosen from the side chains of L- and D-amino acids so that the carbon to which R₂ or R₄ is attached has L or D chirality.

There may also be provided a composition wherein R₁ is —(CH₂)₆—, R₃ is —(CH₂)₈—, and both R₂ and R₄ are the side chain of L-leucine.

More details regarding such polymers and others usable with the present invention are provided in PCT application PCT/US2016/038527 and U.S. patent application Ser. No. 15/188,783, the contents of which is hereby incorporated by reference in its entirety.

There may also be provided a composition further comprising a permeation enhancer.

There may also be provided a composition further comprising one or more compounds selected from the group consisting of polyvinyl alcohol (for example Mowiol 8-88, M_{w} 67 000), dichloromethane, distilled water, hydrocarbons, including alkanes, alkenes, squalene, mineral oil, halogens, alcohols, including glycerols, glycols, polyglycols, ethanol, caprylic alcohol, laury alcohol, organic acids, including oleic acid, undecanoic acid, amines, amides, including pyrrolidone (N-methyl-2-pyrrolidone 2-pyrrolidon)azones, (1-dodecylazacycloheptan-2-one)) urea, isopropyl myristate, sodium lauryl sulfate, cetyltrimethyl ammonium bromide, sorbitan monolaurate, polysorbate 80, dodecyl dimethyl ammoniopropane sulfate, dimethyl sulfoxide, dodecyl methyl sulfoxide, phospholipids, water (for example 70:30, v/v), and carbopol

There may also be provided a composition wherein the formulation is for administration across at least one of dermal and epidermal barriers, and wherein the nanospheres are configured so as to not enter systemic circulation.

There may also be provided a composition wherein the polymer is degraded in-vivo over a period of from about 12 hours to about 48 hours, for example 24 hours.

There may also be provided a composition wherein the nanospheres are between 100 nm and 500 nm in diameter, for example with a mean diameter of about 200 nm.

There may also be provided a composition comprising an emulsifier, for example Kolliphor P188.

There may also be provided a composition further comprising polysorbate 80.

There may also be provided a composition wherein the NSAID forms between 5% and 10% w/w of the composition.

Advantageously, the proposed compositions can provide a relatively large uptake and permeation through the stratum corneum, for example up to (200 μg/cm²).

The proposed nanosphere size enables release of the active ingredient in the dermis, while preventing penetration of the nanoparticles in the circulatory system (which occurs typically with particle size smaller than 100 nm). Free NSAID will target sensory afferents in glabrous skin whilst nanoparticles will move through the hair shaft and release the active targeting hair nociceptors and sensory afferents in the dermis. The proposed nanospheres can also be stored over relatively long period of time after freeze-drying.

In another broad aspect, there is provided a composition comprising at least one Non-Steroidal Anti-Inflammatory Drug (NSAID) encapsulated in biodegradable polymeric nanospheres, wherein the biodegradable polymeric nanospheres include a polymer selected from (1) a poly (ester amide urea) wherein at least one diol, at least one diacid, and at least one amino acid are linked together through an ester bond, an amide bond, and a urea bond, (2) a poly (ester urethane urea) wherein at least one diol and at least one amino acid are linked together through an ester bond, a urethane bond, and a urea bond, (3) a poly (ester amide urethane urea) wherein at least one diol, at least one diacid, and at least one amino acid are linked together through an ester bond, an amide bond, a urethane bond, and a urea bond, (4) a poly (ester amide urethane) wherein at least one diol, at least one diacid, and at least one amino acid are linked together through an ester bond, an amide bond, and a urethane bond, (5) a poly (ester urea) wherein at least one diol and at least one amino acid are linked together through an ester bond and a urea bond, and (6) a poly (ester urethane) wherein at least one diol and at least one amino acid are linked together through an ester bond and a urethane bond, further wherein the at least one diol is a compound of formula:

HO—R₁—OH, R₁ is chosen from C₂-C₁₂ alkylene optionally interrupted by at least one oxygen, C₃-C₈ cycloalkylene, C₃-C₁₀ cycloalkylalkylene,

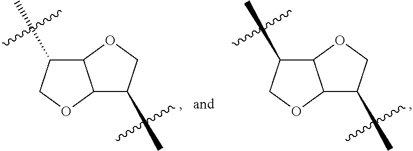

the at least one diacid is a compound of formula:

HO—(CO)—R₃—(CO)—OH,R₃ is C₂-C₁₂alkylene,

the at least one amino acid is chosen from naturally occurring amino acids and non-naturally occurring amino acid.

There may also be provided a composition wherein the polymer is a poly (ester amide urea) (PEAU) comprising the following two blocks with random distribution thereof:

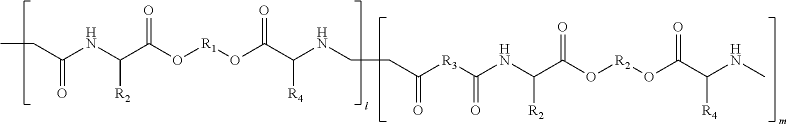

wherein
the ratio of l:m ranges from 0.05:0.95 to 0.95:0.05, l+m=1,
R₁ is chosen from C₂-C₁₂ alkylenes optionally interrupted by at least one oxygen, C₃-C₈ cycloalkylenes, C₃-C₁₀ cycloalkylalkylenes,

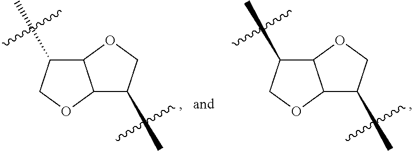

R₃ is C₂-C₁₂ alkylene,
R₂ and R₄ are independently chosen from the side chains of L- and D-amino acids so that the carbon to which R₂ or R₄ is attached has L or D chirality.

There may also be provided a composition wherein R₁ is —(CH₂)₆—, R₃ is —(CH₂)₈—, and both R₂ and R₄ are the side chain of L-leucine.

There may also be provided a composition further comprising a permeation enhancer.

There may also be provided a composition wherein the composition is for administration across at least one of dermal and epidermal barriers, and wherein the nanospheres are configured so as to not enter systemic circulation.

There may also be provided a composition wherein the nanospheres are between 100 nm and 500 nm in diameter.

There may also be provided a composition wherein the nanospheres have a mean diameter of between 100 and 400 nm.

There may also be provided a composition wherein the nanospheres have a mean diameter of between 150 and 200 nm.

There may also be provided a composition wherein the NSAID forms between 5% and 10% w/w of the composition.

There may also be provided a composition wherein the NSAID is ibuprofen.

There may also be provided a composition wherein the composition includes a surfactant and a co-surfactant.

There may also be provided a composition wherein the surfactant is polyvinyl alcohol (PVA) and the co-surfactant is polysorbate 80.

There may also be provided a composition wherein v/v concentrations of PVA and polysorbate 80 in the composition are respectively between 5% and 15% and between 1% and 4%.

There may also be provided a composition wherein v/v concentrations of PVA and polysorbate 80 in the composition are respectively between 9% and 11% and between 1.5% and 2.5%.

In another broad aspect, there is provided a method for manufacturing a suspension of nanospheres containing a non-steroidal anti-inflammatory drug (NSAID), the method comprising:
dissolving the NSAID in a hydrophobic phase, wherein the hydrophobic phase includes a polymer dissolved in an organic solvent, the polymer being selected from
(1) a poly (ester amide urea) wherein at least one diol, at least one diacid, and at least one amino acid are linked together through an ester bond, an amide bond, and a urea bond,
(2) a poly (ester urethane urea) wherein at least one diol and at least one amino acid are linked together through an ester bond, a urethane bond, and a urea bond,
(3) a poly (ester amide urethane urea) wherein at least one diol, at least one diacid, and at least one amino acid are linked together through an ester bond, an amide bond, a urethane bond, and a urea bond,
(4) a poly (ester amide urethane) wherein at least one diol, at least one diacid, and at least one amino acid are linked together through an ester bond, an amide bond, and a urethane bond,
(5) a poly (ester urea) wherein at least one diol and at least one amino acid are linked together through an ester bond and a urea bond, and
(6) a poly (ester urethane) wherein at least one diol and at least one amino acid are linked together through an ester bond and a urethane bond,
wherein
the at least one diol is a compound of formula:
HO—R₁—OH, R₁ is chosen from C₂-C₁₂ alkylene optionally interrupted by at least one oxygen, C₃-C₈ cycloalkylene, C₃-C₁₀ cycloalkylalkylene,

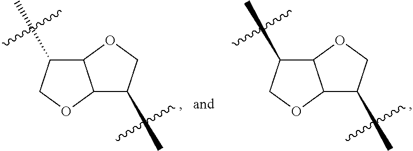

the at least one diacid is a compound of formula:

HO—(CO)—R₃—(CO)—OH,R₃ is C₂-C₁₂alkylene,
the at least one amino acid is chosen from naturally occurring amino acids and non-naturally occurring amino acid;
   emulsifying the hydrophobic phase in a hydrophilic phase; and
   evaporating the organic solvent.

There may also be provided a method wherein the hydrophilic phase includes a surfactant and a co-surfactant.

There may also be provided a method wherein the surfactant is polyvinyl alcohol (PVA) and the co-surfactant is polysorbate 80.

There may also be provided a method wherein v/v concentrations of PVA and polysorbate 80 in combined hydrophilic and hydrophilic phases are respectively between 5% and 15% and between 1% and 4%.

There may also be provided a method further comprising adding additional polysorbate 80 after evaporation of the organic solvent.

There may also be provided a method wherein the solvent is dichloromethane.

There may also be provided a method wherein the polymer is a poly (ester amide urea) (PEAU) comprising the following two blocks with random distribution thereof:

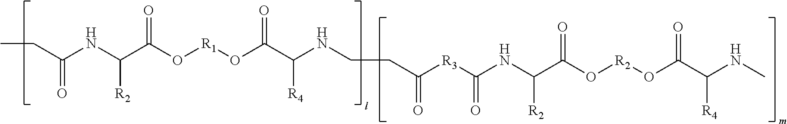

wherein
the ratio of l:m ranges from 0.05:0.95 to 0.95:0.05, l+m=1, R₁ is —(CH₂)₆—, R₃ is —(CH₂)₈—, and both R₂ and R₄ are the side chain of L-leucine.

Other objects, advantages and features of the present invention will become more apparent upon reading of the following non-restrictive description of preferred embodiments thereof, given by way of example only with reference to the accompanying drawings.

### BRIEF DESCRIPTION OF THE DRAWINGS

In the drawings:

FIG. 1 is a scanning electron microscopy image of formulation F1 described in the example below;

FIG. 2 is a scanning electron microscopy image of formulation F9 described in the example below;

FIG. 3 is a scanning electron microscopy image of formulation F9 described in the example below better illustrating the formed nanospheres;

FIG. 4 illustrates the influence of the concentration of polysorbate 80, also known as Tween 80™, on the dimension of nanospheres in a specific embodiment of the present invention both in presence and absence of ibuprofen in the composition; and

FIG. 5 illustrates the influence of the concentration of polysorbate 80, also known as Tween 80™, on the polydispersity index (PDI) of nanospheres in a specific embodiment of the present invention both in presence and absence of ibuprofen in the composition.

### DETAILED DESCRIPTION

A method of topical and/or transdermal delivery of NSAIDs, such as ibuprophen, is proposed, along with compositions suitable for performing this delivery, and methods for manufacturing these compositions. Topical and transdermal delivery offer multiple advantages over other routes of administration. The proposed method allows in some embodiments for the delivery of the NSAIDs in the management of diverse conditions in user-friendly dosage forms that ensure sustained and consistent delivery of efficient doses in the management of medical conditions for which they are found effective. Moreover, topical and transdermal dosage forms promote adherence and eliminate the need for frequent drug consumption.

NSAIDS present a variety of side effects when taken for extended periods of time or in overdosage. Common side effects for regular ibuprofen intake are observed in the gastrointestinal tract, the kidney the coagulation system and the central nervous system. Peptic ulcers are the most prevalent with an estimate of 1 in 5 patients occurrence in chronic users.

The administration of a drug and its efficacy are impacted by more than the nature of the drug or the administration method. The drug must be allowed to reach its clinical target and have as little interaction as possible with nontargets along the way. In topical ibuprofen's case, it is to reduce the ibuprofen in the blood to limit non-specific organ exposure. To aid in this targeted delivery, nanoparticular systems have made their appearance in the last decade. Since then, a great number of methods have been devised using a variety of macromolecules and inorganic compounds.

Current utilization of ibuprofen in topical application is limited. The few marketed options available all make use of alcohol-based gels. This allows to remove the sebaceous oily layer that covers the skin so that the active molecules diffuse into the skin, rather than stay in the sebum due to the hydrophobic nature of most NSAIDs. This layer is then reproduced by the sebaceous glands over the course of a few hours. However, repeated usage, like what is required in arthritic patients, constantly strips the skin of the protection given by the sebum. This leads to a dryness of the skin in the affected area which can cause dermatitis and increased risks of infections. The invention presented does not make use of alcohol to penetrate stratum corneum and diffuses into skin due to the nanoparticles' small size and polarity. It offers a safer and non-irritating (long term) alternative to regular ibuprofen consumers.

In term of regulation, FDA has already put in place a system for non-prescription use through Rx-to-OTC Switch Process. FDA already approved non-prescription Voltaren Arthritis Pain™ to GlaxoSmithKline plc and of non-prescription Pataday Twice Daily Relief™ and Pataday Once Daily Relief™ to Alcon. The Office of Non-prescription Drugs in the FDA's Center for Drug Evaluation and Research set framework for approval of a wider range of non-prescription drugs with the aim of providing the millions of people that suffer with joint pain from arthritis daily over-the-counter access to another non-opioid treatment option. Evidently, data must be provided to demonstrate that the drug is safe and effective for use in self-medication as directed in proposed labeling. NSAIDs were shown to have a certain cardiac effect, notably in increases of heart attacks, heart failures and strokes. However, these effects are rare and often the blame is shared by other comorbidities, especially in older individuals. By decreasing the total systemic bioavailable dosage, it would decrease the exposition of the drug in the organism all the while offering an efficacy comparable to oral counterparts but in the affected locations rather than the whole body.

Voltaren Arthritis Pain, which contains diclofenac and is intended for the temporary relief of joint pain, is not for immediate relief and may take up to 7 days to work. In the delivery system proposed in the present document, NSAIDs, such as ibuprofen are entrapped in biodegradable polymers, such as a polyester amide urea polymer, for optimized prolonged topical delivery and permeability of ibuprofen for pain and inflammation. This delivery method has high potential for rapid pain relief as well as an anti-inflammatory effect in the form of an easily absorbed, penetrating gel or cream. Application could include but not limited to muscle pain, back pain, joint pain and arthritic pain.

Current scientific works have obtained mitigated success in making polymeric nano vehicles for delivery of NSAIDs. Notably, Llera-Rojas et al. (1) have developed polymeric biodegradable ibuprofen-loaded nanospheres. Their nanospheres were smaller (183 nm) and obtained a decent zeta potential (20.9 mV). However, their preparation used 250 mg of ibuprofen in a 300 mL volume and their encapsulation efficiency was 43%. This means they had extremely low concentration of active drug (about 20 times less) compared to the proposed PEAU formulation (0.83 mg/ml vs 18.18 mg/mL). Another group, Pham et al., (2) have designed ibuprofen-loaded solid lipid nanoparticules (SLNs) in an hydrogel for topical application. Their nanoparticles were significantly smaller (98 nm) but they had poor stability (3.17 mV) and similar encapsulation efficiency (58%).

The proposed Ibuprofen-PEAU nanoparticles present numerous advantages. Being composed of Amino acid-based biodegradable polymers (AABBPs), they are entirely composed of non-toxic building blocks such as naturally occurring amino acids and fatty diols and dicarboxylic acids. These compounds contain hydrolysable ester bonds at a monomer stage, which when incorporated into the polymeric backbones are responsible for the biodegradation of the polymers. These have evident advantages over biodegradable polyesters (PEs) (e.g. polyglycolic and poly (lactic acids)) and their copolymers as polycondensation synthesis can occur without any toxic catalyst, they possess higher hyophilicity and, hence, better compatibility with tissues as well as longer shelf-life. They also present a variable hydrophobicity/hydrophilicity balance suitable for constructing devices suitable for sustained/controlled drug release and an erosive mechanism and in vitro biodegradation rates ranging from 10⁻³ to 10⁻¹ mg/(cm²·h) that can be regulated by the addition of enzymes. Their biodegradation is complete and even and they present excellent adhesion to plastic metal and glass surfaces. Polyester amides are also known to be biocompatible, hemocompatible, and are used in stent-based local drug delivery. Additionally, it has been shown that polyester degradation by-products are acidic, resulting in undesirable side-effects at a cellular level, which limits their use as functional tissue engineering scaffolds. In contrast to polyesters, AABBPs degradation biproducts are less acidic and more biocompatible. For example, hydrolysis products of poly (ester amide) s are neutral (zwitterionic) amino acids, readily metabolizable diols, and weak fatty acids. Another example are the biodegradation products of poly (ester urethane) s and poly (ester urea) s which are naturally occurring physiological compounds such as CO₂, hydrophobic amino acids, and diols.

The advantages of nanoparticles are also notable as they improve solubility and bioavailability of the drug. They are cost-effective to produce and provide a stable recipient for the entrapped drug. They lower drug-associated toxicity and protect the active agent from unnecessary degradation while allowing for a sustained release of active agents at optimum therapeutic concentrations. Additionally, they may offer site-specific targeted delivery of the drug, improving absorption in afflicted areas. What is more, PEAU delivery system and its innate properties drive the cartilage regeneration process by improving the wound environment, i.e. PEAU utilizes enzymes in the wound environment to biodegrade, thus reducing protease concentrations, reducing the negative impact on articular regeneration, while providing a sustained, prolonged release of ibuprofen at the site of injury. Other similar polymers, such as the polymers mentioned in the Summary of invention section are also usable to manufacture the nanospheres.

It should be noted that the claimed invention was achieved only after numerous failures. The proposed polymer had been used previously to form particles mainly as water in oil in water double emulsions, to encapsulate bacteriophages and other compounds. Since this was the main manufacturing process to produce particles, nanocapsules were first tried. These nanocapsules encapsulate an aqueous phase inside polymeric shells, which are themselves suspended in another aqueous phase. If workable, this approach would ensure that suitable doses of ibuprofen could be delivered, as one could obtain high concentrations of ibuprofen in the encapsulated phase. Since ibuprofen is highly hydrophobic, a direct approach in which ibuprofen would be in aqueous solution before encapsulation was not possible. First, the use of an organic solvent in the internal phase was tried, to solubilize ibuprofen. However, such solvents prevented the formation of nanocapsules, most likely because the polymer used is also soluble in organic solvents. Then, oleic acid (OA) was tried as the main component of the internal phase. OA is a fatty acid, and ibuprofen is relatively soluble in OA (about 300 mg/mL). OA is also used in cosmetics, and regarded as safe for topical application. While nanocapsules were obtained, it was not possible to isolate them from the OA phase after being created.

After all these failures, it was decided to produce nanospheres. During the manufacturing process, the polymer is dissolved in an organic solvent, for example dichloromethane (DCM), along with the ibuprofen, which is then dispersed in an aqueous phase to form the nanospheres, which are typically in majority, or entirely, full, made of a continuous polymer phase. Such polymer nanospheres are advantageous compared to liposomes and emulsions used in the prior art to deliver ibuprofen. Indeed, these latter formulations have difficulty penetrating the skin due to interaction with the skin surface.

It should be noted that simply incorporating ibuprofen in DCM during manufacture of nanocapsules led initially to unsatisfactory results, and other compounds were required during the manufacturing process to achieve a suitable encapsulation in high enough doses, with nanoparticles having suitable size distributions, as detailed below.

The invention proposes the following:

Potential candidates for entrapped NSAIDs include but are not limited to: diclofenac, diflunisal, etodolac, fenoprofen, flurbiprofen, ibuprofen, indomethacin, ketoprofen, ketorolac, mefenamic acid, meloxicam, nabumetone, naproxen, oxaprozin, piroxicam, sulindac, tolmetin and coxibs.

Excipients include polyvinyl alcohol (Mowiol 8-88, M_{w} 67 000), dichloromethane (used for polymer solubilization and then evaporated in its entirety) and distilled water. Other non-limiting excipients include permeation enhancers selected from the group consisting of hydrocarbons, including alkanes, alkenes, squalene, mineral oil, halogens, alcohols, including glycerols, glycols, polyglycols, ethanol, caprylic alcohol, lauryl alcohol, organic acids, including oleic acid, undecanoic acid, amines, amides, including pyrrolidone (N-methyl-2-pyrrolidone 2-pyrrolidon)azones, (1-dodecylazacycloheptan-2-one)) urea, isopropyl myristate, sodium lauryl sulfate, cetyltrimethyl ammonium bromide, sorbitan monolaurate, polysorbate 80, dodecyl dimethyl ammoniopropane sulfate, dimethyl sulfoxide, dodecyl methyl sulfoxide, phospholipids, water (for example 70:30, v/v), and carbopol. In other embodiments, permeation enhancers are not used.

Afflictions that would benefit from the invention could include but are not limited to: ankylosing spondylitis, antiphospholipid antibody syndrome, autoimmune encephalitis, chronic recurrent multifocal osteomyelitis, gout, Henoch-Schonlein purpura, juvenile dermatomyositis, juvenile idiopathic arthritis, juvenile lupus (SLE), juvenile scleroderma, juvenile vasculitis, Kawasaki disease, lupus (Systemic Lupus Erythematosus), mixed connective tissue disease, myositis, poststreptococcal inflammatory syndromes, psoriatic arthritis, reactive arthritis, rheumatoid arthritis, scleroderma, Sjogren's syndrome, spondyloarthritis/spondyloarthropathy, systemic juvenile idiopathic Arthritis, undifferentiated connective tissue disease, uveitis, vasculitis.

### EXAMPLES

To make ibuprofen (IBU)-PEAU nanoparticles, the following protocol was used:

### Materials:

Fisherbrand™ 150 Handheld Homogenizer Motor
Fisherbrand™ 150 Hand Held Homogenizer Accessory, Stainless Steel Probe
100 mL glass beaker
Glass lens
Low recipient to serve as ice water bath for beaker
Pipette gun
10 mL and 25 mL pipettes
PVA solution (10%)
PEAU solution (12.5 or 25% in DCM)
Stirring bar
Stirring Block
50 mL Falcon tubes
4-Isobutyl-alpha-methylphenylacetic acid, 99% (Ibuprofen powder)

### Method:

### PEAU-IBU Solution

In a 50 mL Falcon tube, weigh 1000 mg of ibuprofen
Using a pipette, add 10 mL of 25% PEAU solution making sure to catch any powder that gets stuck on the sides of the tube
Homogenize at 35 000 rpm in ice cold water for 1 min (E1)

### Dispersant

In 100 mL beaker with a stirring bar, add 30 ml of PVA 10%

### Emulsion

Place the PVA solution beaker in an ice cold bath
Start the homogenizer at 35 000 rpm in the PVA solution
Slowly pour in the PEAU-IBU solution and move the beaker in circular motions in order to mix the 2 phases
Add 2% v/v polysorbate 80 concentration
Keep homogenizing for 10 min (E2) then place the beaker on a stirring plate, cover with a glass lens and stir at maximum speed without causing splash for 18 h.
   Stability agent and storage
Collect the resulting formulation in 50 mL Falcon tubes using a pipette before adding 500 uL of polysorbate 80, for a 2% polysorbate 80 concentration
Store at 4° C.

Analysis for HPLC were done using the following:

### Sample Preparation

Vortex samples thoroughly (1 min)
In duplicate, weight 20-30 mg of formulation in an eppendorf tube
Add 200 uL H2O, vortex
Centrifuge 11 000 rpm 5 min
Remove supernatant
Add 200 uL H2O, vortex
Centrifuge 11 000 rpm 5 min
Remove supernatant
Add 100 uL DCM, vortex
Add 100 uL ACN, vortex
Add 100 uL of MeOH
Add 900 uL (MeOH:ACN:H2O) 50:30:20, vortex
Sonicate 20 min
Centrifuge 11 000 rpm 5 minutes
Dilute 10 uL+990 uL (MeOH:ACN:H2O) 50:30:20, vortex
Inject 2 uL

### LC-MS Method

Instrument: Agilent 1200 coupled to TOF MS 6224
Mobile phase A: 0.15 mM NH4F in H2O
Mobile phase B: ACN
Column: Poroshell EC-C18 4.6×100 mm, 2.7 um
ESI negative
SIM mode: m/z 204.97

Analysis for DLS and zeta potential were done using a Malvern Zetasizer and following method:

### Sample Preparation

Dilute formulation 1/200 in distilled water and vortex until solution looks turbid but monophasic Run for DLS and zeta potential

There are 2 formulations who were kept for they showed good potential, these being F1 and F9. Formulation F1 had the following conditions:

| Formulation | Stirring | PEAU | | E1 | E2 | IBU mass |
|---|---|---|---|---|---|---|
| Name | speed(rpm) | % | PVA % | (min) | (min) | (g) |
| F1 | 35 000 | 12.5 | 10 | 1 | 10 | 0.5 |

And gave the following results:

| | Normal | | IBU(g) per | | | Zeta |
|---|---|---|---|---|---|---|
| Formulation | distribution | [IBU] | gram of | Average | Polysorbate | potential |
| Name | (Y/N)?* | (mg/ml) | PEAU | size (nm) | 80% | (mV) |
| F1 | Y | 6.5 | 0.527 | 227 | 2 | −30.5 |

*Note:
Formulations were checked for the homogeneity of their nanoparticle population because it was discovered that there were sometimes multiple populations within a size distribution.

Formulation F9 had the following conditions

| Formulation | Stirring | PEAU | | E1 | E2 | IBU mass |
|---|---|---|---|---|---|---|
| Name | speed(rpm) | % | PVA % | (min) | (min) | (g) |
| F9 | 35 000 | 25 | 10 | 1 | 10 | 0.5 |

And gave the following results:

| | Normal | | IBU(g) per | | | Zeta |
|---|---|---|---|---|---|---|
| Formulation | distribution | [IBU] | gram of | Average | Polysorbate | potential |
| Name | (Y/N)?* | (mg/ml) | PEAU | size (nm) | 80% | (mV) |
| F9 | Y | 20.1 | 0.693 | 215 | 2 | −28.7 |

Scanning electron microscopy (SEM) was also effectuated on F1 and F9. The following method was used to prepare the sample for analysis:
Dilute formulation sample 1/10 in distilled water. Vortex
Centrifuge at 10 000 rpm/10 min
Remove supernatant before resuspending in 1 mL of distilled water
Repeat washing step until supernatant is practically clear
Suspend nanoparticle pellet in 1 mL of distilled water.
Drop 10 uL of diluted nanopaticule solution on cut pieces of aluminium foil (1×1 cm)
Leave to dry at room temperature until completely dry (˜3-4 hours)

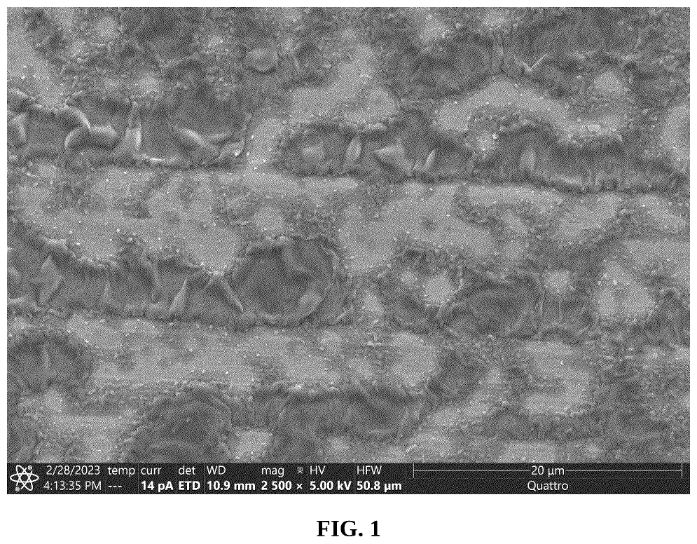

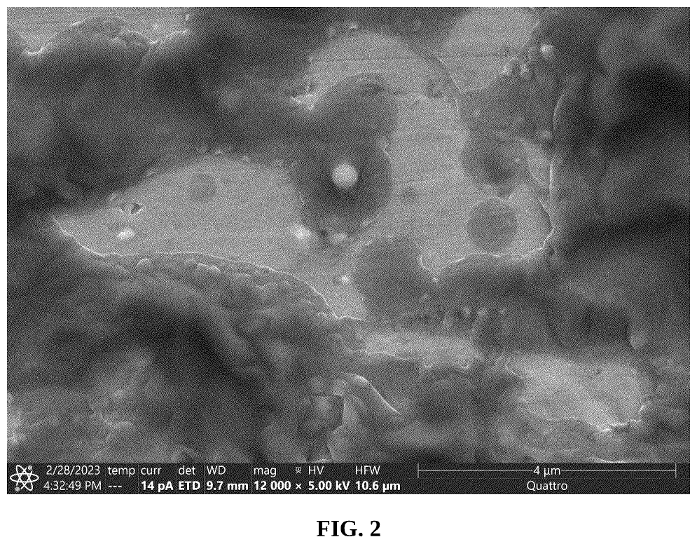

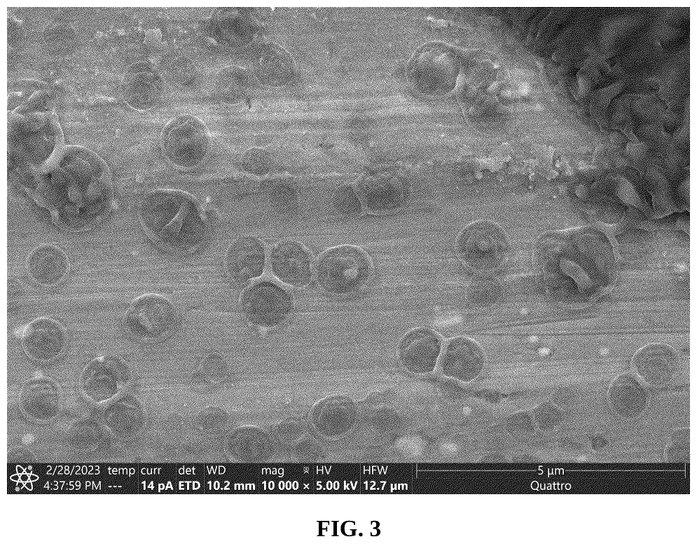

Results (FIGS. 1 to 3) shows PVA (the gooey dark grey substance) as a residual contaminant of the washes, the aluminum foil which serves as the background and appears as light grey and finally, the nanospheres which appear as tiny white specs.

In the proposed composition, ibuprofen and other NSAID molecules are trapped in a solid PEAU matrix. Thus, IBU-PEAU nanospheres are prepared using a single emulsion (oil-water). The nanospheres are suspended typically in a relatively high concentration PVA solution, for example about 10% PVA. This high amount of PVA is advantageous as nanosized particles are relatively unstable, and PVA is a know stabilizing agent for colloidal suspensions.

The kinetics of release for the active compound in nanospheres differs from the kinetics of release in nanocapsules, as in the former case, the compound is released gradually, while in the latter case, all of the compound is released once the envelope of the capsules is breached.

It is noted that the proposed composition is relatively difficult to achieve successfully, as the NSAID is, before being incorporated to the polymer, a powder. Furthermore, an unexpected synergy between ibuprofen and polysorbate 80 was observed, as detailed below.

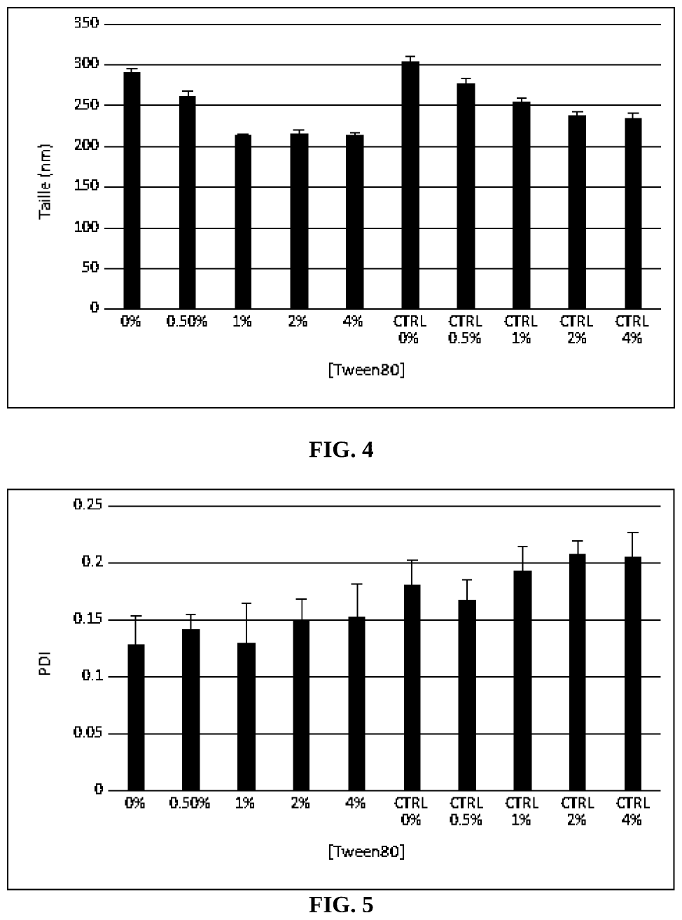

FIG. 4 illustrates the average diameter of nanospheres of a formulation similar to formulation F1 described above, but with varying concentrations of polysorbate 80 (denoted Tween 80™ in the figure) in the presence of ibuprofen (left bars), or in controls, in which ibuprofen was omitted (right bars, CRTL). Surprisingly, ibuprofen and polysorbate 80 synergistically interact to reduce nanosphere diameter, and nanospheres of about 200 nm in diameter were obtainable. Smaller nanospheres are desirable, as they enhance permeation of the composition through the skin, which is required to reach the joints or muscle tissue to treat with ibuprofen. Of note, nanospheres that would be much smaller than 100 nm would be undesirable, as these nanospheres would then be able to enter systemic circulation, and possibly cause side effects that one wishes to avoid using topical application. Further evidence for this synergy can be found in FIG. 5, where it is shown that not only the ibuprofen-polysorbate 80 interaction produce smaller than expected nanospheres, but it also produces compositions in which the nanospheres have more homogeneous size distribution, as measured by the polydispersity index (PDI), which is smaller in the ibuprofen containing nanospheres than in the same compositions omitting ibuprofen.

Yet furthermore, it was observed that polysorbate 80 greatly increased the encapsulation efficiency of the F1 formulation, from 42% in the absence of polysornate 80 to 78% in its presence. Surprisingly, the effect of polysorbate 80 on encapsulation efficiency presented in the literature is the opposite of the observed effect. Indeed, Sharma et al., observed that increasing polysorbate 80 concentration in the preparation of poly (lactic-co-glycolic acid) PLGA nanospheres loaded with paclitaxel reduced encapsulation efficiency. The opposite effect was observed in the present system. (3).

### REFERENCES

Llera-Rojas, V. G., Hernández-Salgado, M., Quintanar-Guerrero, D., Leyva-Gómez, G., Mendoza-Elvira, S., Villalobos-García, R., Llera-Rojas, V. G., Hernández-Salgado, M., Quintanar-Guerrero, D., Leyva-Gómez, G., Mendoza-Elvira, S., & Villalobos-García, R. (2019). Comparative study of the release profiles of ibuprofen from polymeric nanocapsules and nanospheres. Journal of the Mexican Chemical Society, 63 (2), 61-70.
Pham, C. V., Van, M. C., Thi, H. P., Thanh, C. Ð., Ngoc, B. T., Van, B. N., Le Thien, G., Van, B. N., & Nguyen, C. N. (2020). Development of ibuprofen-loaded solid lipid nanoparticle-based hydrogels for enhanced in vitro dermal permeation and in vivo topical anti-inflammatory activity. Journal of Drug Delivery Science and Technology, 57, 101758.
Sharma, N., Madan, P., & Lin, S. (2016). Effect of process and formulation vari-ables on the preparation of parenteral paclitaxel-loaded biodegradable polymeric nanoparticles: A co-surfactant study. Asian Journal of Pharmaceutical Sciences, 11 (3), 404-416.

Although the present invention has been described hereinabove by way of exemplary embodiments thereof, it will be readily appreciated that many modifications are possible in the exemplary embodiments without materially departing from the novel teachings and advantages of this invention. Accordingly, the scope of the claims should not be limited by the exemplary embodiments, but should be given the broadest interpretation consistent with the description as a whole. The present invention can also be modified, without departing from the spirit and nature of the subject invention as defined in the appended claims.

What is claimed is:

1. A composition comprising at least one Non-Steroidal Anti-Inflammatory Drug (NSAID) encapsulated in biodegradable polymeric nanospheres, wherein the biodegradable polymeric nanospheres include a polymer selected from
(1) a poly (ester amide urea) wherein at least one diol, at least one diacid, and at least one amino acid are linked together through an ester bond, an amide bond, and a urea bond,
(2) a poly (ester urethane urea) wherein at least one diol and at least one amino acid are linked together through an ester bond, a urethane bond, and a urea bond,
(3) a poly (ester amide urethane urea) wherein at least one diol, at least one diacid, and at least one amino acid are linked together through an ester bond, an amide bond, a urethane bond, and a urea bond,
(4) a poly (ester amide urethane) wherein at least one diol, at least one diacid, and at least one amino acid are linked together through an ester bond, an amide bond, and a urethane bond,
(5) a poly (ester urea) wherein at least one diol and at least one amino acid are linked together through an ester bond and a urea bond, and
(6) a poly (ester urethane) wherein at least one diol and at least one amino acid are linked together through an ester bond and a urethane bond,
further wherein
the at least one diol is a compound of formula:
HO—R₁—OH, R₁ is chosen from C₂-C₁₂ alkylene optionally interrupted by at least one oxygen, C₃-C₈ cycloalkylene, C₃-C₁₀ cycloalkylalkylene, 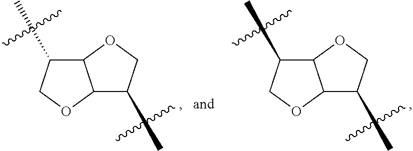
the at least one diacid is a compound of formula:
HO—(CO)—R₃—(CO)—OH,R₃ is C₂-C₁₂alkylene,
the at least one amino acid is chosen from naturally occurring amino acids and non-naturally occurring amino acid.

2. The composition as defined in claim 1, wherein the polymer is a poly (ester amide urea) (PEAU) comprising the following two blocks with random distribution thereof: 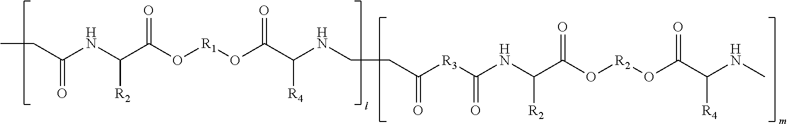
wherein
the ratio of l:m ranges from 0.05:0.95 to 0.95:0.05, l+m=1,
R₁ is chosen from C₂-C₁₂ alkylenes optionally interrupted by at least one oxygen, C₃-C₈ cycloalkylenes, C₃-C₁₀ cycloalkylalkylenes, 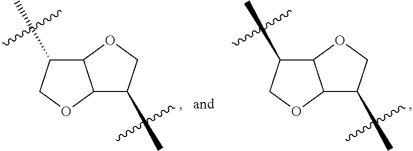
R₃ is C₂-C₁₂ alkylene,
R₂ and R₄ are independently chosen from the side chains of L- and D-amino acids so that the carbon to which R₂ or R₄ is attached has L or D chirality.

3. The composition as defined in claim 2, wherein R₁ is —(CH₂)₆—, R₃ is —(CH₂)₈—, and both R₂ and R₄ are the side chain of L-leucine.

4. The composition as defined in claim 1, further comprising a permeation enhancer.

5. The composition as defined in claim 1, wherein the composition is for administration across at least one of dermal and epidermal barriers, and wherein the nanospheres are configured so as to not enter systemic circulation.

6. The composition as defined in claim 1, wherein the nanospheres are between 100 nm and 500 nm in diameter.

7. The composition as defined in claim 1, wherein the nanospheres have a mean diameter of between 100 and 400 nm.

8. The composition as defined in claim 1, wherein the nanospheres have a mean diameter of between 150 and 200 nm.

9. The composition as defined in claim 1, wherein the NSAID forms between 5% and 10% w/w of the composition.

10. The composition as defined in claim 1, wherein the NSAID is ibuprofen.

11. The composition as defined in claim 1, wherein the composition includes a surfactant and a co-surfactant.

12. The composition as defined in claim 10, wherein the surfactant is polyvinyl alcohol (PVA) and the co-surfactant is polysorbate 80.

13. The composition as defined in claim 12, wherein v/v concentrations of PVA and polysorbate 80 in the composition are respectively between 5% and 15% and between 1% and 4%.

14. The composition as defined in claim 12, wherein v/v concentrations of PVA and polysorbate 80 in the composition are respectively between 9% and 11% and between 1.5% and 2.5%.

15. A method for manufacturing a suspension of nanospheres containing a non-steroidal anti-inflammatory drug (NSAID), the method comprising:
dissolving the NSAID in a hydrophobic phase, wherein the hydrophobic phase includes a polymer dissolved in an organic solvent, the polymer being selected from
(1) a poly (ester amide urea) wherein at least one diol, at least one diacid, and at least one amino acid are linked together through an ester bond, an amide bond, and a urea bond,
(2) a poly (ester urethane urea) wherein at least one diol and at least one amino acid are linked together through an ester bond, a urethane bond, and a urea bond,
(3) a poly (ester amide urethane urea) wherein at least one diol, at least one diacid, and at least one amino acid are linked together through an ester bond, an amide bond, a urethane bond, and a urea bond,
(4) a poly (ester amide urethane) wherein at least one diol, at least one diacid, and at least one amino acid are linked together through an ester bond, an amide bond, and a urethane bond,
(5) a poly (ester urea) wherein at least one diol and at least one amino acid are linked together through an ester bond and a urea bond, and
(6) a poly (ester urethane) wherein at least one diol and at least one amino acid are linked together through an ester bond and a urethane bond,
wherein
the at least one diol is a compound of formula:
HO—R₁—OH, R₁ is chosen from C₂-C₁₂ alkylene optionally interrupted by at least one oxygen, C₃-C₈ cycloalkylene, C₃-C₁₀ cycloalkylalkylene, 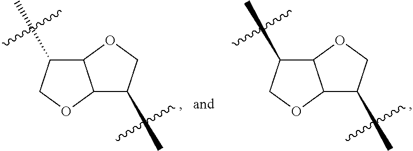
the at least one diacid is a compound of formula:
HO—(CO)—R₃—(CO)—OH,R₃ is C₂-C₁₂alkylene,
the at least one amino acid is chosen from naturally occurring amino acids and non-naturally occurring amino acid;
emulsifying the hydrophobic phase in a hydrophilic phase; and
evaporating the organic solvent.

16. The method as defined in claim 15, wherein the hydrophilic phase includes a surfactant and a co-surfactant.

17. The method as defined in claim 16, wherein the surfactant is polyvinyl alcohol (PVA) and the co-surfactant is polysorbate 80.

18. The method as defined in claim 17, wherein v/v concentrations of PVA and polysorbate 80 in combined hydrophilic and hydrophilic phases are respectively between 5% and 15% and between 1% and 4%.

19. The method as defined in claim 18, further comprising adding additional polysorbate 80 after evaporation of the organic solvent.

20. The method as defined in claim 15, wherein the solvent is dichloromethane.

21. The method as defined in claim 15, wherein the polymer is a poly (ester amide urea) (PEAU) comprising the following two blocks with random distribution thereof: 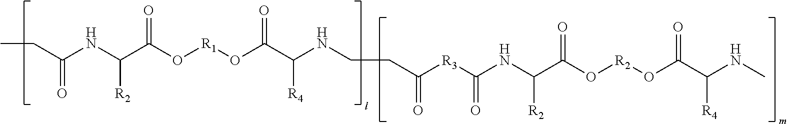
wherein
the ratio of l:m ranges from 0.05:0.95 to 0.95:0.05, l+m=1, R₁ is —(CH₂)₆—, R₃ is —(CH₂)₈—, and both R₂ and R₄ are the side chain of L-leucine.
